(19) United States
(12) Patent Application Publication
(10) Pub. No.: US 2022/0054387 A1
(43) Pub. Date: Feb. 24, 2022

(54) COMPOSITION COMPRISING AT LEAST ONE GLYCOSYLATED FLAVONOID AND ITS USE IN COSMETICS OR DERMATOLOGY

(71) Applicant: PURALLY, Orleans (FR)

(72) Inventor: Gérard REDZINIAK, Antony (FR)

(73) Assignee: PURALLY, Orleans (FR)

(21) Appl. No.: 17/386,641

(22) Filed: Jul. 28, 2021

(30) Foreign Application Priority Data
Aug. 21, 2020 (FR) 2008601

(51) Int. Cl. A61K 8/60; A61Q 19/00

(52) U.S. Cl. CPC A61K 8/602; A61K 2800/75; A61Q 19/00

(57) ABSTRACT

The present invention relates to a cosmetic and/or dermatological composition comprising as an active ingredient at least one glycosylated flavonoid, characterized in that it further comprises, as an active ingredient, a compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene and/or a compound characterized by its ability to absorb blue light.

### TECHNICAL FIELD

The present invention relates to compositions comprising at least one glycosylated flavonoid, which can be used in topical applications, in the treatment of non-allergic pathologies that trigger the production of IL31, in particular pathologies affecting the skin and chosen among pruritus, atopic dermatitis, eczema, psoriasis, and nodular prurigo.

### PRIOR ART

Skin is the main barrier that protects the human body from the environment. This environment is composed of exogenous factors that can cause physical, chemical or biological stress, which increases skin aging and the risk of developing skin conditions with varying degrees of severity. It is now accepted that pollution and sunlight have a synergistic and negative effect on the skin (The skin aging exposome, Krutmann J. et al. Dermatol Sci. 2017 March; 85(3):152-161).

There are various types of pollution: air pollution, water pollution, global warming (thermal pollution), chemical pollution from industries or even from the components in everyday objects (paint, solvent, glue, etc.).

Sunlight, which is made up of visible light, UVAs/UVBs, but also blue light, leads to the production of inflammatory IL-1, IL-6, IL-8 cytokines. Other recent studies show that blue light, which is emitted by the sun but also by all light from devices such as computers, tablets, televisions and cell phones, has an influence on photoaging, as it increases the level of internal free radicals in keratinocytes and melanocytes. This also leads to the formation of dark spots (Duteil et al, Differences in visible light-induced pigmentation according to wavelengths: a clinical and histological study in comparison with UVB exposure, Pigment Cell Melanoma Res, 2014 vol. 27(5) pp. 822-6 T).

Air pollution also plays an important role in skin pathologies, in particular the polycyclic aromatic hydrocarbons (PAHs) that are given off by burning wood but especially, in urban environments, by exhaust gases. In a recent study, researchers showed, on human skin explants, that PAH metabolization leads to the production of easily-eliminated metabolites but that a small fraction reacts with DNA to induce mutations.

This study also shows that exposure to solar UVs reduces the skin cells' ability to metabolize PAHs, which could thus accumulate in tissues (Metabolism and genotoxicity of polycyclic aromatic hydrocarbons in human skin explants: mixture effects and modulation by sunlight, Anne von Koschembahr et al., Archives of Toxicology volume 94, pages 495-507 (2020); Solar simulated light exposure alters metabolization and genotoxicity induced by benzo[a]pyrene in human skin, Anne von Koschembahr et al., Scientific Reports volume 8, Article number: 14692 (2018)).

The decrease in metabolism may lead to an accumulation of benzo[a]pyrene (B[a]P) and toxicity over the long-term.

Some skin pathologies do not have an inflammatory component, for example, pruritus which is nevertheless characterized by the secretion of certain cytokines such as IL-31 (Interleukin 31) (IL-31 is crucial for induction of pruritus, but not inflammation, in contact hypersensitivity, Ayako Takamori et al., Sci Rep (2018) 8:6639).

IL-31 is a cytokine in the IL-6 family and is produced by activated CD4+ T lymphocytes, suggesting its involvement in the development of a Th2 immune response. It has been shown that mice which are transgenic for IL-31 develop skin inflammations of atopic dermatitis type. Moreover, 11-31 seems to be the cause of pruritus.

There are different ways to treat itching, especially itching caused by an increase in IL-31 production. Therapeutic antibodies against IL-31 have been developed and are used in the treatment of pruritus or atopic dermatitis. One example is Nemolizumab, an antibody developed by Galderma in clinical trials in moderate to severe atopic dermatitis. U.S. Pat. No. 10,273,297 and EP2734549 describe the use of other anti-IL-31 antibodies in this field of application. Nevertheless, therapeutic antibody treatments are not without side effects, since they are not specific to the IL-31 produced by keratinocytes in the skin. These treatments are also expensive. There is thus a real need for a solution that makes it possible to mitigate the production of IL-31 in keratinocytes by means of localized treatment.

The inventors first observed that the production of IL-31 by human keratinocytes was increased after exposure to PAHs in synergy with exposure to blue light.

The inventors have, surprisingly, demonstrated that glycosylated flavonoid compounds make it possible to reduce the production of IL-31 by keratinocytes, induced by exposure to PAHs and blue light.

### Technical Problem to be Solved

One of the purposes of the present invention is to provide cosmetic and/or dermatological compositions that make it possible to reduce the production of IL-31 in the skin and to treat or prevent a pathology affecting the skin, chosen among pruritus, atopic dermatitis, eczema, psoriasis, and nodular prurigo.

### DETAILED DESCRIPTION OF THE INVENTION

One of the purposes of the present invention is to provide a cosmetic and/or dermatological composition comprising as an active ingredient at least one glycosylated flavonoid, characterized in that it further comprises, as an active ingredient, a compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene and/or a compound characterized by its ability to absorb blue light.

“Cosmetic composition” refers to any substance or composition intended to be in contact with the various surface areas of the human body (epidermis, hair and body hair systems, nails, lips and external genitals) or with the teeth and oral mucosa, with an exclusive or essential view to cleansing and perfuming them, altering their appearance, protecting them, keeping them in good condition or correcting bodily odors.

“Dermatological composition” refers to any substance or composition intended to treat or prevent skin conditions or for regular skin care.

“Active ingredient” refers to a natural or chemical substance, extracted or synthetic, which is included in the formulation of a therapeutic or cosmetic composition, characterized in that it has a beneficial, therapeutic or preventive effect on a pathology or symptom. “Flavonoid” refers to any compound in the flavonoid family, characterized by the same base structure formed by two aromatic rings bound by three carbons: C6-C3-C6, divided into the following classes: flavones, flavonols, flavononols or dihydroflavonols, flavanones, aurones, chalcones and dihydrochalcones.

“Glycosylated flavonoid” refers to any compound belonging to the classes described above, carrying at least one sugar chosen among glucose, galactose and rhamnose.

The flavonoids according to the invention are natural or synthetic flavonoids. Flavonoids are naturally present in fruit, vegetables, tea, and wine, as well as in certain medicinal plants such as hawthorn, ginkgo, passion flower, peppermint, artichokes or neem and bitter melons, but also in many other plants and flowers (non-exhaustive list). They can be extracted. Many methods have been published and are accessible to the person skilled in the art.

In one embodiment of the invention, the composition according to the invention further comprises, as an active ingredient, a compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene. “Limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene” refers to the ability of any substance to keep PAH-type pollutants on the outer surface of the skin. In other words, limiting or preventing the penetration of a PAH into keratinocytes means keeping the keratinocytes in their physiologically-balanced state, i.e., when the structure and functions are not altered, that they do not have inflammatory characteristics or markers such as the synthesis and release of cytokines such as TNF alpha, for example (The influence of PM2.5 on lung injury and cytokines in mice. Yang J, et al., Exp Ther Med. 2019 October; 18(4):2503-2511). This extremely aggressive interleukin causes the skin's membrane and cells to deteriorate, which leads to skin inflammation and aging (Wang Y., et al., Mech Ageing Dev. 2019 December, 184:111160).

The polycyclic aromatic hydrocarbons, also referred to as PAHs, are benzo[a]pyrene (C₂₀H₁₂), cyclopenta[c,d]pyrene (C₁₈H₁₀), dibenzo[a,h]anthracene (C₂₂H₁₄) or dibenzo[a,l]pyrene (C₂₄H₁₈ or C₂₄H₁₄) or derivatives of these compounds. These compounds are generated in gaseous or particulate form by the combustion of fossil fuels (in particular by diesel engines). PAHs are therefore particularly present in urban air pollution.

Hydrocarbons enter the human body by inhalation, by ingestion, but also by transfer through the skin. Indeed, the inventors have shown in studies conducted prior to this invention that exposure of human keratinocytes to PAHs increased the production of IL-31 in the keratinocytes.

In one embodiment of the invention, the composition according to the invention can also comprise a compound characterized by its ability to absorb blue light.

“Ability to absorb blue light” refers to the ability of any substance to prevent the negative effects of blue light on skin aging and skin alteration, by limiting the skin's exposure to said blue light.

Blue light, which ranges between wavelengths of 400 to 500 nanometers (nm), is part of the visible light spectrum which is comprised between 400 and 800 nm. It is emitted by solar irradiation but also by all electronic devices with a screen using Electroluminescent Diodes (also called LEDs), such as smartphones, tablets, televisions or computers.

The energy emitted by this blue light can reach almost 40 J/cm² for solar irradiation and 10 J/cm² for the screens of electronic devices.

In one preferred embodiment, the glycosylated flavonoid is a glycosylated flavonol. Flavonols make up a class of flavonoids characterized by a C3 phenol group and a C═O C4 carbonyl group on the central heterocyclic ring of the base flavonoid skeleton. Glycosylated flavonols can be in the form of mono-, di- or triglycosides. The monoglycosides include astragalin (kaempferol containing a glucose in the R3 group), azalein (azaleatin containing a rhamnose in the R3 group), hyperoside (quercetin containing a galactose in the R3 group), isoquercitin (quercetin containing a glucose in the R3 group), myricitrin (myricetin containing a rhamnose in the R3 group), quercitrin (quercetin containing a rhamnose in the R3 group), rutoside (quercetin containing a rutinose in the R3 group), xanthorhamnin (rhamnetin containing a trisaccharide in the R3 group), amurensine (kaempferol containing a glucose in the R7 group and a tert-amyl alcohol in the R8 group), spiraeoside (quercetin containing a glucose in the R4′ [sic: R4] group) and troxerutin (quercetin containing a rutinose in the R3 group and 3 hydroxyethyl.)

The diglycosides include kaempferitrin (kaempferol containing a rhamnose in the R3 group and a rhamnose in the R7 group), robinin (kaempferol containing a robinose in the R3 group and a rhamnose in the R7 group), icariin (kaempferide containing a rhamnose in the R3 group and a glucose in the R4 group and a prenyl group and a methoxyl group.)

In one preferred embodiment, the glycosylated flavonoid is rhamnosylated or glucosylated, preferably rhamnosylated.

In one preferred embodiment, the rhamnosylated flavonoid is chosen among myricitrin (5,7-dihydroxy-3-[(2S,3R,4R,5R,6S)-3,4,5-trihydroxy-6-methyloxan-2-yl]oxy-2-(3,4,5-trihydroxyphenyl)chromen-4-one), quercitrin (2-(3,4-dihydroxyphenyl)-5,7-dihydroxy-3-[[(2S,3R,4R,5R,6S)-3,4,5-trihydroxy-6-methyl-2-tetrahydropyranyl]oxy]-4-chromenone), kaempferitrin (C₂₇H₃₀O₁₄), azalein (2-(3,4-dihydroxyphényl)-7-hydroxy-5-méthoxy-3-[(2S,3R,4R,5R,6S)-3,4,5-trihydroxy-6-methyloxan-2-yl]oxychromèn-4-one), icariin (5-Hydroxy-2-(4-methoxyphenyl)-8-(3-methylbut-2-enyl)-7-[(2S,3R,4S,5S,6R)-3,4,5-trihydroxy-6-(hydroxymethyl)oxan-2-yl]oxy-3-[(2S,3R,4R,5R,6S)-3,4,5-trihydroxy-6-methyloxan-2-yl]oxychromen-4-one) or a mixture of at least two of these compounds. The mixture can be composed of myricitrin and quercitrin or of myricitrin and kaempferitrin or of myricitrin and azalein or of myricitrin and icariin or of quercitrin and azalein or of quercitrin and icariin or of azalein and icariin. The mixture may also be composed of three rhamnosylated flavonoids such as myricitrin, quercitrin and azalein or myricitrin, quercitrin and icariin or myricitrin, kaempferitrin and azalein or myricitrin, azalein and icariin or laxine, for example. The list is not exhaustive.

In one preferred embodiment, the rhamnosylated flavonoid is myricitrin.

In one embodiment of the invention, the compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene is chosen among polysaccharides, cyclic polysaccharides such as cyclodextrine, emulsifiers, lyophilized hyaluronic acid.

Skin consists of different layers and of different cell types. The outermost layer, the epidermis, is composed of keratinocytes, melanocytes and Langerhans cells. This epidermis is divided into four layers characterized by the maturity level of the keratinocytes of which they are composed. From the innermost to the outermost, the layers are: the basal layer, the Malpighi layer, the granular layer and the stratum corneum. This stratum corneum is a hydrophobic barrier that acts as the necessary barrier to protect the skin.

Given the hydrophobic nature of the stratum corneum, lipophilic substances such as polycyclic aromatic hydrocarbons can easily penetrate into the keratinocytes. Polysaccharides, also referred to as glycans, polyosides, polyholosides or complex carbohydrates, are polymers in the carbohydrate family which are made up of several monosaccharides that are bonded together by osidic bonds.

Polysaccharides have hydrophilic properties. As a result, and due to the hydrophobic nature of PAHs, these polysaccharides create a hydrophilic physical barrier against the penetration of these substances.

The polysaccharides used in the context of the invention can be cyclic polysaccharides such as cyclodextrine that include alpha-cyclodextrine (α-cyclodextrine, C₃₆H₆₀O₃₀), beta-cyclodextrine (β-cyclodextrine, C₄₂H₇₀O₃₅), gamma-cyclodextrine (γ-cyclodextrine, C₄₈H₈₀O₄₀.) Cyclodextrines are known to form a molecular cage that encapsulates various molecules. They are widely used in the agri-food and pharmaceutical industries. Their natural origin is an advantage. In the context of this invention, cyclic polysaccharides such as cyclodextrin participate in the capture of PAHs by means of their “hydrophobic cage” molecular structure in the core of the cyclic form of the glucose polymer (6 to 9 residuals.)

Cyclodextrines, which are hydrophobic on the inside and hydrophilic on the outside, capture PAH-type molecules and create a molecular trap for said PAHs.

Thus, one of the subject-matters of the invention is a composition comprising, as an active ingredient, at least one glycosylated flavonoid, more particularly myricitrin, quercitrin, azalein, icariin or a mixture of at least two of these compounds and a cyclodextrin.

In the context of the invention, emulsifiers are compounds capable of limiting or preventing the penetration into the keratinocytes of a polycyclic aromatic hydrocarbon. An emulsifier is a compound capable of creating an emulsion. All emulsifiers have a hydrophilic end and a hydrophobic end, which makes it possible to mix hydrophilic and hydrophobic substances. Polycyclic aromatic hydrocarbons are by nature lipophilic and hydrophobic.

The purpose of adding a cyclodextrine compound to the composition of the invention is to create an emulsion between a PAH or a mixture of PAHs and a hydrophilic compound, in order to limit or prevent penetration into the keratinocytes of said PAHs.

The emulsifiers that can be used in the context of the composition of the invention are: lecithin, phospholipids, lanolin or beeswax (natural emulsifiers), glycerol stearate, Polysorbate 60 and the PEG-6/PEG32/Glycol Stearate blend marketed under the name Tefose® 63, glycolipids such as sophorolipids or octylglucoside from saponins, lipoproteins (non-exhaustive and non-limiting list).

Thus, one of the subject-matters of the invention is a composition comprising, as an active ingredient, at least one glycosylated flavonoid, more particularly myricitrin, quercitrin, azalein, icariin or a mixture of at least two of these compounds and an emulsifier.

In the context of the invention, lyophilized hyaluronic acid is also a compound capable of limiting or preventing the penetration into the keratinocytes of a polycyclic aromatic hydrocarbon.

Lyophilized hyaluronic acid has lipophilic and hydrophilic amphiphilic properties as an emulsifying base. This molecule captures lipophilic molecules such as B[a]P in the fatty chains that make up the lyophilized part.

Thus, one of the subject-matters of the invention is a composition comprising, as an active ingredient, at least one glycosylated flavonoid, more particularly myricitrin, quercitrin, azalein, icariin or a mixture of at least two of these compounds and lyophilized hyaluronic acid.

In one embodiment of the invention, the compound capable of absorbing blue light is chosen among carotenoids or melanoidins.

Carotenoids encompass the molecules in the carotene and xanthophyll families. The main carotenoids are astaxanthin, lycopene, beta-carotene, lutein and zeaxanthin. Carotenoids are liposoluble and have recognized antioxidant and photoprotection properties. Carotenoids can therefore be used in the composition according to the invention due to their ability to absorb blue light (Blue-Violet Light Irradiation Dose Dependently Decreases Carotenoids in Human Skin, which Indicates the Generation of Free Radicals. Vandersee S. et al., Oxidative Med Cell Longev. Volume 2015, Article ID 579675).

Thus, one of the subject-matters of the invention is a composition comprising, as an active ingredient, at least one glycosylated flavonoid, more particularly myricitrin, quercitrin, azalein, icariin or a mixture of at least two of these compounds and a cyclodextrin or an emulsifier or lyophilized hyaluronic acid, and a carotinoid.

Melanoidins are heterogeneous brown polymers with high molecular weight that form during the Maillard reaction, by combining sugars and amino acids. Due to their structure and chromophorous properties (absorbent in wavelengths of from 400 to 500 nm), they are good blue light filters. (Coffee melanoidins: structures, mechanisms of formation and potential health impacts, Moreira A S, et al., Food Funct. 2012 September; 3(9):903-15).

Thus, one of the subject-matters of the invention is a composition comprising, as an active ingredient, at least one glycosylated flavonoid, more particularly myricitrin, quercitrin, azalein, icariin or a mixture of at least two of these compounds and a cyclodextrin or an emulsifier or lyophilized hyaluronic acid, and a compound in the melanoidin family.

In one embodiment of the invention, the composition of the invention is formulated for topical application.

The compositions that can be used in the invention can be in any of the dosage forms typically used for topical application and in particular in the form of an aqueous, alcoholic or hydroalcoholic solution or suspension or of an oily suspension or of a solution or a dispersion such as a lotion or serum or of a liquid or semi-liquid emulsion such as a milk, obtained by dispersion of a fatty phase in an aqueous phase (O/W) or inversely (W/O) or of a soft-textured suspension or emulsion such as a cream (O/W) or (W/O) or of an aqueous or anhydrous gel, of an ointment, of a loose or compact powder to be used as is or to be incorporated into an excipient, or in the form of microcapsules or microparticles or of ionic and/or nonionic vesicular dispersions.

In a known manner, the composition according to the invention can also contain admixtures that are typically used in the cosmetic, pharmaceutical or dermatological field, such as hydrophilic or lipophilic gelling agents, hydrophilic or lipophilic active ingredients, preservatives, antioxidants, solvents, fragrances, fillers, filters, bactericides, odor absorbers and colorants. The person skilled in the art knows how to choose the admixtures and their quantities according to the desired dosage form.

In one preferred embodiment of the invention, the composition according to the invention is formulated as a mist as described in Example 8.

In one embodiment, the invention relates to a glycosylated flavonoid compound, preferably a rhamnosylated flavonol, for use in the treatment of non-allergic pathologies that trigger the production of IL31, in particular pathologies affecting the skin and chosen among pruritus, atopic dermatitis, eczema, psoriasis, and nodular prurigo.

“Treatment” refers to the attenuation or disappearance of at least one symptom of the pathology, which can be a decrease in the production of IL-31 by the keratinocytes, a decrease in the urge to scratch, a decrease in skin inflammation, up to the disappearance of the symptom(s).

The inventors have shown that exposure of human keratinocytes to PAHs and/or blue light leads to an increase in the production of IL-31 by said keratinocytes. Moreover, some authors have shown that the itchy skin in cases of pruritus or atopic dermatitis was associated with this production of IL-31 (New mechanism underlying IL-31-induced atopic dermatitis, Meng J. el [sic: et] al., J Allergy Clin Immunol. 2018 May; 141(5):1677-1689; IL-31: A new key player in dermatology and beyond, Ba\check{g}ci I S et al., J Allergy Clin Immunol. 2018 March; 141(3):858-866). One of the subject-matters of the invention relates to a glycosylated flavonoid compound, preferably a rhamnosylated flavonol, for use in the treatment of non-allergic pathologies that trigger the production of IL-31, in particular pathologies affecting the skin and chosen among pruritus, atopic dermatitis, eczema, psoriasis, and nodular prurigo.

Pruritus is clinically defined as the subjective and unpleasant sensation of an urge to scratch. There is a sensory component beginning in the skin, which runs through the dorsal ganglia and is processed in the central nervous system. Often, the motor reaction follows in the form of an urge to scratch. Scratching damages the skin and triggers an inflammatory reaction, which further increases the itch.

Atopic dermatitis, also called atopic eczema, is a chronic inflammatory skin disease. It develops preferentially in infants and children, but may persist, or sometimes even appear, in adolescents and adults. It is characterized by dry skin (xerosis cutis) associated with eczema-type lesions (redness and itching, blisters, oozing and crusts) that develop in flare-ups.

Eczema is a syndrome, a set of clinical signs and symptoms common to several conditions, which may correspond to several inflammatory skin diseases. These diseases are characterized by itching (pruritus), congestive redness of the skin (erythema), and skin rash.

Psoriasis is a chronic inflammatory disease characterized by well-defined, red, raised plaques covered in whitish scales or by a thin silvery scurf that detaches easily.

Prurigo or nodular prurigo is a dermatosis (skin disease) characterized by pruriginous (itchy) lesions, some of which are peeling and/or associated with scratch marks, accompanied by severe itching that may disrupt sleep. Scratching sustains the lesions, which become crusted. Acute prurigos (lasting less than 6 weeks), which are most often secondary to insect bites, are distinguished from chronic prurigos that may be of dermatological, neurological, psychiatric or systemic origin.

In one preferred embodiment of the invention, the rhamnosylated flavonol used in the treatment of non-allergic pathologies that trigger the production of IL31, in particular pathologies affecting the skin and chosen among pruritus, atopic dermatitis, eczema, psoriasis, nodular prurigo, is chosen among myricitrin, quercitrin, kaempferitrin, azalein, icariin or a mixture or at least two of these compounds. The mixture can be composed of myricitrin and quercitrin or of myricitrin and azalein or of myricitrin and icariin or of quercitrin and azalein or of quercitrin and icariin or of azalein and icariin. The mixture can also be composed of three rhamnosylated flavonoids, such as myricitrin, quercitrin and azalein or myricitrin, quercitrin and icariin or myricitrin, azalein and icariin or quercitrin, azalein and icariin.

### DESCRIPTION OF THE FIGURES

FIG. 1 presents a cell viability assay that measures the percentage of viable cells of human keratinocytes placed in the presence of myricitrin at different doses;

FIG. 2 presents the effect of benzo[a]pyrene, alone or in combination with blue light, on the production of IL-31 by human keratinocytes. Exposure to blue light is expressed in Joules per square centimeter (J/cm².) Exposure to benzo[a]pyrene is expressed in micromoles (μμM);

FIG. 3 presents the effect, in pg/ml, of myricitrin on the release of IL-31 in keratinocytes that have been previously exposed to benzo[a]pyrene and blue light;

FIG. 4 presents the concentrations of IL31, standardized by the amount of total cellular proteins, illustrating the effects of astaxanthin alone compared to the effects of myricitrin alone, after exposure of keratinocytes to blue light and to benzo[a]pyrene;

FIG. 5 presents the inhibition value of the release of IL31 for two concentrations of myricitrin and astaxanthin.

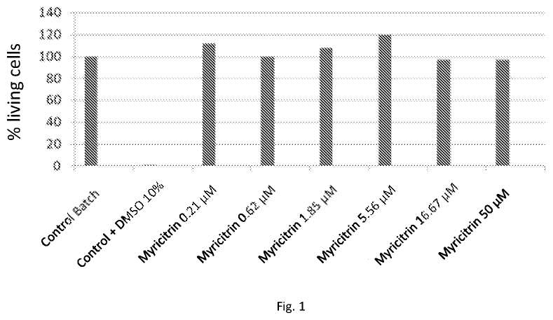

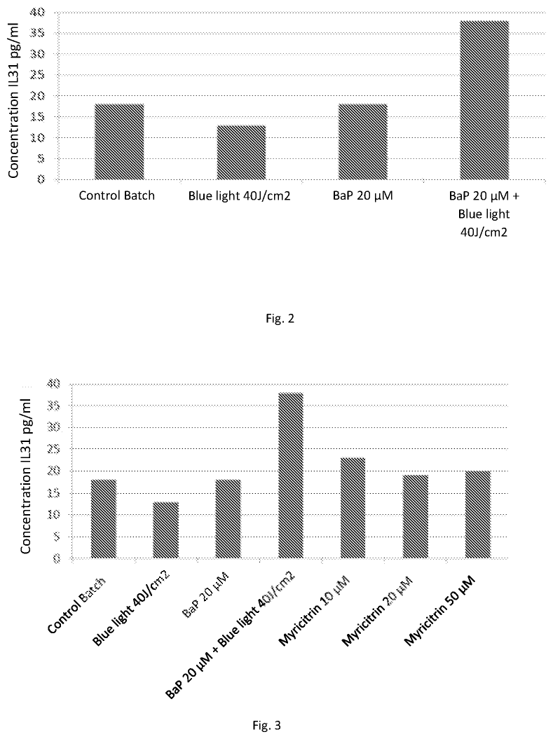

### EXAMPLES

### Example 1: Culture of Human Keratinocytes

The cells used in this study are primary cultures of normal human keratinocytes (NHKs) extracted after skin surgery of a 30-year-old donor.

They were cultured in a complete KSFM environment: Keratinocyte-SFM (17005-31, Gibco) with L-Glutamine, supplemented with a recombinant human epidermal growth factor (EGFhr, 10450-013, Gibco), extract of bovine pituitary gland (BPE, 13028-014, Gibco) and 1% of streptomycin penicillin (15070-063, Gibco) at 37° C. in 5% CO₂ atmosphere up until 80% confluence.

### Example 2: Keratinocyte Viability Assay

On D0, normal human keratinocytes are incubated in 96-well plates in a culture medium. The cells are left to stabilize for 24 hours at 37° C. in 5% CO₂ atmosphere, as described in Example 1.

On D1, the cells are treated with myricitrin dissolved in the culture medium at different concentrations.

The culture is kept for 24 hours at 37° C. and 5% CO₂.

The NHK viability was assessed using a Cell Proliferation Kit II (XTT) (11465015001, Roche) in accordance with the manufacturer's instructions.

The XTT system, a colorimetric method, is a test used to quantify mitochondrial activity. This method, which is simple, accurate and allows for reproducible results, can be used as a viability assay. This assay is based on the breakdown of yellow XTT tetrazolium salt into orange formazan by the “succinate-tetrazolium reductase” system present in the cells' mitochondrial respiratory chain. Conversion thus only takes place in metabolically active cells, which means living cells.

The formazan derivate is measured by spectrophotometry (at 450 nm with 650 nm of reference.) For each condition, optical density data averages (OD, absorbance) are calculated.

The viability of the treated cells is expressed as a percentage of untreated control batch: a treatment that reduces the viability of the cells to below the comparative limit of 80% of mitochondrial activity in the untreated control batch, is considered cytotoxic for the cells. Inversely, an increase in the data is a sign of mitochondrial activity and possibly even a sign of cell proliferation.

Keratinocytes cultured according to Example 1 are exposed to a range of myricitrin of from 0.21 to 50 μμM. The Control batch represents the culture medium with no admixture. A 10% control+DMSO represents a toxic medium, indicating cell toxicity. Experience shows that myricitrin has no toxic effect on keratinocytes.

### Example 3: Use of a Flavonoid such as a Rhamnosylated Flavonol on Cultured Keratinocytes

The keratinocytes cultured according to Example 1 are exposed either to benzo[a]pyrene (B[a]P) at 20 μμM, or to blue light (450 nm) at 40 J/cm² or to the combination of both at the same doses. A myricitrin treatment with amounts ranging from 10 μμM to 50 μμM is applied to cells that have previously received the B[a]P and blue light combination.

After 24 hours' treatment, an assay of the interleukin 31 (IL-31) released by the cells is performed, via an immunoassay kit (ELISA.) The IL-31 assay was performed with the human IL-31 ELISA MAX™ Deluxe Set (445704, Biolegend.)

The combination of benzo[a]pyrene and blue light induces a +111% increase in IL-31 by the keratinocytes. The experiment shows that adding myricitrin as of 20 μM induces a −100% decrease in the production of IL-31 on cells pre-treated with 20 μM of B[a]P and 40 J/cm² of blue light.

### Example 4: Use of a Flavonoid such as Rhamnosylated Flavonol in Patients

In a group of volunteers working in urban areas [Caucasian and Asian women aged 20 to 60] and presenting with problems of cutaneous discomfort on the face (itching, pruritus, etc.), the cream in Example 5 is applied on one side of the face and a placebo cream with no myricitrin is applied on the other side of the face.

From the time of application and over eight days of morning and evening use, the volunteers record their organoleptic experience—freshness, gentleness and ease of application of the products—along with their physiological sensations such as calmness and relief from itching, on a questionnaire. Statistical analyses are performed at the end of the assay to demonstrate the efficiency of the product containing myricitrin.

### Example 5: Calming and Softening Cream for Urban Skin or Skin Prone to Itching

A—Aqueous Phase

Glycerin 2.0%, Hexylene Glycol 3.0%, Xanthan Gum 0.5%, Myricitrin 0.5%, Preservatives qs (sufficient quantity), Carbomer 0.35%, NaOH 0.35%, Water qsp. 100%

B—Fatty Phase

Squalane 15%, Cetyl Alcohol 2%, Arachidyl Alcohol/Behenyl Alcohol/Arachidyl Glucoside 1%, Glycerol Stearate 5%, Preservative and fragrance qs

### Example 6: Soothing and Moisturizing Cream for Urban Skin Prone to Atopic Dermatitis

A—Fatty Phase

Ceteareth-2 3.5%, Ceteareth-21 between 2 and 4%, Wheat Germ Oil 3%, Cyclomethicone 7%, Octyl Palmitate 8%

B—Aqueous Phase

Glycerin 7.0%, Hexylene Glycol 3.0%, Preservatives qs, Water qsp. 100%

C—Ingredients Added to the Emulsion, at a Temperature Below 40° C.

Sodium Hyaluronate 0.1%, Myricitrin 1.0%, Water 5%, Tocopherol 0.05%, Vitamin A Palmitate 0.1%, Phospholipids 0.5%, Ceramides 3 0.1%, Polyacrylamide & C14-13 Isoparaffin & Laureth-7 between 2 to 3.5%

### Example 7: Cream and Milk for Skin Exposed to Blue Light or with Dark Spots

A—Fatty Phase

Glycerol Monostearate 2%, PEG-100 Stearate 3%, C12-C15 Alkyl Benzoate 10%, Dimethicone 5%, Tocopherol Acetate 1%, Octyl-Triazone (Uvinul T150) 1.5%, Butyl Methoxy Dibenzoyl Methane (Eusolex 9020) 2.0%, Ceostearyl Alcohol 1%

B—Aqueous Phase

Water qsp. 100%, Quercitrin 0.5%, Preservatives 0.6%, Glycerin 7%, Hexylene Glycol 3.0%, Carbomer 0.5%, Tetra Sodium EDTA 0.2%, Sodium Hyaluronate 0.1%, NaOH 0.5%, Preservative+fragrance qs.

C—Ingredients Added to the Emulsion, at a Temperature Below 40° C.

Tocopherol Acetate between 0.1 and 1%, Pyridoxine between 0.01 and 0.05%, Vitamin A Palmitate between 0.01 and 1%, d-Panthenol between 0.1 and 1%, Citric Acid between 0.1 and 0.5%, Zinc Gluconate between 0.1 and 1%, Trisodium Citrate between 1 and 2.5%, Water 5%.

### Example 8: Protective Mist for Skin Exposed to Blue Light from Electronic Devices

Water qsp. 100%, Kaempferitrin 2%, Preservatives 0.6%, Glycerin 7%, Hexylene Glycol 3.0%, Carbomer 0.5%, Tetra sodium EDTA 0.2%, Sodium Hyaluronate 0.1%, NaOH 0.5%, Preservative+fragrance qs.

### Example 9: Comparison of the Effects of Astaxanthin and Myricitrin on the Release of IL31 by Primary Keratinocytes Exposed to the Combined Effects of Blue Light and of benzo[a]pyrene (BaP)

Astaxanthin (dihydroxy-3,3′ dioxo-4,4′ (3-carotene) is a carotenoid synthesized in particular by microalgae. This molecule absorbs wavelengths comprised between 400 nm and 500 nm.

Benzo[a]pyrene (CAS 50-32-8) is one of the most toxic polycyclic aromatic hydrocarbons (PAHs).

The cell treatments were carried out as follows.
a) Cell type: primary keratinocytes from human epidermis (39-year-old donor, ref: KER110, batch KER110049, Biopredic)
b) Culture conditions: 96-well plate, 37° C., 5% CO₂, seeding 20,000 cells/cm²
c) Culture medium: KSFM (Gibco ref: 10144892)+1% penicillin/streptomycin
d) Treatment with the compounds: performed 24 hours after seeding,
e) Incubation time with the compounds: 24 hours of contact
f) Concentrations of the compounds tested: myricitrin and astaxanthin at 20 μM and 50 μM
g) Stress condition: performed following the treatment with the compounds; exposure to irradiation (blue light; 40 J/cm²; 450 nm); followed by 24 hours of treatment with 20 μM of benzo[a]pyrene (CRM40071, Sigma-Aldrich) in culture medium.

The analyses were performed according to the following procedure.

Control batch: no treatment other than the renewal of the culture medium

Samples:
Culture media
Cell pellets

Endpoints/readout:
IL-31 assay, using the culture media (Human IL-31 ELISA MAX Deluxe; Biolegends)
Assay of total proteins, using the cell pellets (Bradford Method)

Results: IL31 Assay

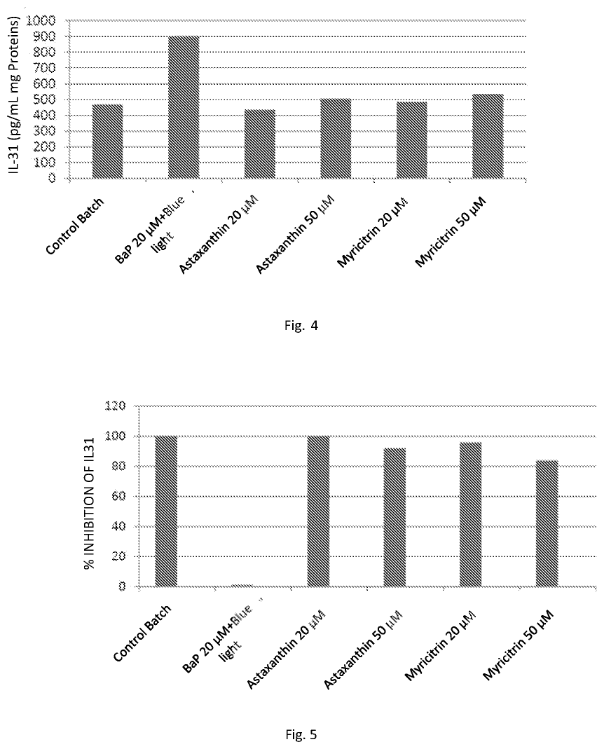

In FIG. 4, the results are expressed as IL-31 concentration normalized by the amount of total cell proteins.

In FIG. 5, a release inhibition value (%) was calculated for the experimental groups, using the following formula:

% IL31 Release Inhibition: % Batch X=(IL31 Stress−IL31 Batch X)/(IL31 Stress−IL31 Control)×100

As a reference, the control batch was examined at maximum efficiency (100%) and the stress group (Blue light+BaP) at minimum efficiency (at 0%):

1. A cosmetic and/or dermatological composition comprising as an active ingredient at least one glycosylated flavonoid, and further comprising, as an active ingredient, a compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene and/or a compound characterized by its ability to absorb blue light.

2. The cosmetic and/or dermatological composition according to claim 1, characterized in that said glycosylated flavonoid is a glycosylated flavonol.

3. The cosmetic and/or dermatological composition according to claim 1, characterized in that said glycosylated flavonoid is rhamnosylated or glycosylated, preferably rhamnosylated.

4. The cosmetic and/or dermatological composition according to claim 3, characterized in that said rhamnosylated flavonoid is chosen among myricitrin, quercitrin, kaempferitrin, azalein, icariin or a mixture of at least two of these compounds.

5. The cosmetic and/or dermatological composition according to claim 4, characterized in that said rhamnosylated flavonoid is myricitrin.

6. The cosmetic and/or dermatological composition according to claim 1, characterized in that said compound capable of limiting or preventing the penetration into the keratinocytes of a polycyclic aromatic hydrocarbon such as benzo[a]pyrene, cyclopenta[c,d]pyrene, dibenzo[a,h]anthracene or dibenzo[a,l]pyrene is chosen among polysaccharides, cyclic polysaccharides, such as cyclodextrin, emulsifiers, lyophilized hyaluronic acid.

7. The cosmetic and/or dermatological composition according to claim 1, characterized in that said compound capable of absorbing blue light is chosen among carotenoids or melanoidins.

8. The cosmetic and/or dermatological composition according to claim 1, characterized in that it is formulated for topical application.

9. A glycosylated flavonoid compound, preferably a rhamnosylated flavonol, for use in the treatment of non-allergic pathologies that trigger the production of IL31, in particular pathologies affecting the skin and chosen among pruritus, atopic dermatitis, eczema, psoriasis and nodular prurigo.

10. A glycosylated flavonoid compound, preferably a rhamnosylated flavonol according to claim 9, chosen among myricitrin, quercitrin, kaempferitrin, azalein, icariin or a mixture of at least two of these compounds.
